(19) United States
(12) Patent Application Publication
(10) Pub. No.: US 2023/0113447 A1
(43) Pub. Date: Apr. 13, 2023

(54) SYSTEM FOR CONTROLLING A RENAL THERAPY DEVICE

(71) Applicant: QIDNI LABS INC., Buffalo, NY (US)

(72) Inventors: Morteza AHMADI, Buffalo, NY (US); Clarence GRAANSMA, Buffalo, NY (US); Raman SUD, Buffalo, NY (US)

(73) Assignee: QIDNI LABS INC., Buffalo, NY (US)

(21) Appl. No.: 17/759,831

(22) PCT Filed: Jan. 29, 2021
(86) PCT No.: PCT/US2021/015784

§ 371 Date: Jul. 29, 2022

Related U.S. Application Data
(63) Continuation-in-part of application No. PCT/US2020/026716, filed on Apr. 3, 2020.
(60) Provisional application No. 62/968,997, filed on Jan. 31, 2020.

(51) Int. Cl. A61M 1/16; H02J 7/00; A61M 60/113; A61M 60/37

(52) U.S. Cl. CPC A61M 1/1603; H02J 7/00032; H02J 7/0013; H02J 7/0047; H02J 7/0063; H02J 7/00712; A61M 60/113; A61M 60/37; H02J 2310/23; A61M 2205/18; A61M 2205/3331; A61M 2205/8206; A61M 1/28

(57) ABSTRACT

A system and method for controlling a renal therapy device is provided. The system comprises a controller for performing a method comprising: receiving a first power level of a first battery to determine if the first power level of the first battery is above a threshold value and receiving a second power level of a second battery to determine if the second power level of the second battery is above the threshold value. When the first power level is above the threshold value, the controller causes the renal therapy device to be powered by the first battery. When the first power level is below the threshold value, and the second power level is above the threshold value, the controller causes the renal therapy device to be powered by the second battery. The first battery is electrically isolated from the second battery.

### CROSS REFERENCE TO RELATED APPLICATION AND CLAIM OF PRIORITY

The present application claims priority to International Application No. PCT/US2020/026716 filed on Apr. 3, 2020, the entire contents of which are hereby incorporated by reference; and to U.S. Provisional Patent Application No. 62/968,997 filed on Jan. 31, 2020, the entire contents of which are hereby incorporated by reference.

### BACKGROUND OF THE INVENTION

### Field of the Invention

The disclosure related generally to fluid processing systems and methods for renal therapy, and more particularly to systems and methods including sorbent for removing toxins and/or water from fluid.

### Background of the Invention

More than 2.5 million patients worldwide utilize some form of dialysis such as hemodialysis (HD) or peritoneal dialysis (PD) as a life-saving treatment. Current dialysis modalities, however, still have many deficiencies in their use as a replacement of the function of normal kidneys. The most important deficiency of current methods of HD is its intermittent nature, resulting in large fluctuations in the internal electrolyte environment and the fluid volume of the patient compared to the regulation achieved by the normal kidney function. PD provides more continuous dialysis, but the clearance of uremic toxins is relatively low compared to HD. Failure of the PD method for patients is frequent over a longer term mostly due to damage caused to the peritoneal membrane by peritonitis infection and the use of high intraperitoneal glucose concentrations that are required for the osmotic fluid removal.

The total effect of the deficiencies of existing renal therapies means that long term survival is much less for these patients compared to the general population. Extended and more frequent treatments can improve survival as well as improve the quality of life of these patients. The use of existing renal therapy technologies in home settings in order to allow for longer and more frequent treatments has had some success at improving outcomes as compared to in-center HD treatment but has been limited by economic factors, logistics issues and limitations of space in patient homes such that only a relatively small percentage of patients are treated with home HD. Another disadvantage of existing home HD treatments is that patients are connected to large medical devices and water treatment systems for extended periods of time, severely affecting mobility. The weight of conventional HD modules is upwards of 60 kg. Also, purification of approximately 120 liters of water per session by additional equipment in a fixed location is needed. This equipment cannot be readily moved to other locations and so limits the home HD patients daily mobility and ability to travel.

### SUMMARY OF THE INVENTION

The disclosure herein is directed to the use of at least one sorption substance or sorption material in a device for the removal of toxic substances and excess water from blood or other biofluids from a patient, and methods of renal therapy involving removing toxic substances from blood and other biofluids using the sorption substance or sorption material suitable for use in said device and in other hemodialysis and peritoneal dialysis systems. The patient can be a human or an animal.

In an aspect, the disclosure describes a sorbent cartridge for use in a portable wearable renal therapy system. The sorbent cartridge comprises: an inlet and an outlet, the inlet configured to receive process fluid from the renal therapy system and the outlet configured to discharge treated process fluid; a hydrogel configured to absorb and adsorb a toxin from the process fluid without use of a dialysate to purify the process fluid. The inlet and the outlet are each configured to releasably couple to the renal therapy device for removing the sorbent cartridge.

In another aspect, the disclosure describes a sorbent cartridge for use in a renal therapy system. The sorbent cartridge comprises a hydrogel configured to absorb or adsorb a toxin from a process fluid and the sorbent cartridge is configured to be releasably coupled to the renal therapy system for ease of removability.

In an embodiment, the hydrogel is configured to release to the process fluid at least one of an electrolyte, a buffer, a mineral, a vitamin, or an anti-coagulant. In an embodiment, the buffer is sodium bicarbonate. In an embodiment, the anti-coagulant is at least one of heparin and citrate.

In an embodiment, the hydrogel is formed as a plurality of beads, the plurality of beads position in a reservoir of the sorbent cartridge configured to receive process fluid flowing through the sorbent cartridge, the sorbent cartridge comprising a filter to prevent passage of the plurality of beads into circulation of the process fluid.

In an embodiment, the sorbent cartridge is configured such that the hydrogel is in direct contact with the process fluid.

In an embodiment, the sorbent cartridge is configured such that the hydrogel is in indirect communication with the process fluid across a membrane.

In an embodiment, the hydrogel is configured to absorb toxins from the process fluid into the hydrogel without altering electrolyte levels outside of a physiological range that would cause harm to a user of the renal therapy system.

In an embodiment, the hydrogel is configured to absorb 1 gram to 100 grams of urea in 24 hours from the process fluid without altering electrolyte levels outside of a physiological range that would cause harm a user of the renal therapy system.

In an embodiment, the hydrogel is configured to absorb electrolytes into the hydrogel to reduce specific electrolyte levels of the process fluid.

In an embodiment, the sorbent cartridge is configured to be releasably coupled to a renal therapy system, where the renal therapy system is a portable wearable system.

In an embodiment, the sorbent cartridge comprises a first compartment and a second compartment, the first compartment comprising a membrane configured to remove water from the process fluid, and the second compartment comprising the membrane configured to toxin removal.

In an embodiment, the hydrogel is cast in place over the membrane in said sorption cartridge, and wherein the membrane is a hollow fiber membrane.

In an embodiment, the sorbent cartridge comprises a temperature sensor, and at least one of a heating element and/or a cooling element, the temperature sensor configured to send a temperature signal to a controller, and the at least one of a heating element and/or a cooling element configured to receive a output signal from a controller.

In an embodiment, the sorbent cartridge comprises a conductive member configured to couple with a cooling element to create a temperature gradient along a distance between the conductive member and the membrane.

In an embodiment, the sorbent cartridge comprises a vibration element configured to vibrate the hydrogel.

In an embodiment, the hydrogel forms a hydrogel layer having a thickness of greater than or equal to about 1 mm.

In an embodiment, the hydrogel forms a hydrogel layer having a thickness of between 1 mm and 3 mm.

In an embodiment, the hydrogel forms a hydrogel layer having a thickness of is greater than or equal to 3 mm.

Embodiments may include combinations of the above features.

In another aspect, the disclosure describes a renal therapy system comprising the sorbent cartridge of any one of the embodiments of the sorbent cartridges described above. The renal therapy system is at least one of a hemodialysis system, a peritoneal dialysis system, a hemoperfusion system, a hemofiltration system, or a hemodiafiltration system.

In an embodiment, the system is a portable wearable system.

In an embodiment, the renal therapy system comprises a cooling element to create a temperature gradient along a length of the hydrogel.

In an embodiment, the renal therapy system comprises a vibration element configured to vibrate the hydrogel.

Embodiments may include combinations of the above features.

In another aspect, the disclosure describes a use of the sorbent cartridge of any one of the sorbent cartridges described above for renal therapy of a user.

In another aspect, the disclosure describes a method for removing toxic substances from process fluid. The method comprises: providing a sorbent cartridge comprising a hydrogel; moving process fluid through the sorbent cartridge in communication with the hydrogel, the process fluid comprising toxins; and absorbing or adsorbing the toxins from the process fluid into the hydrogel to provide treated process fluid.

In an embodiment, the method comprises heating the process fluid to about 37° C.

In an embodiment, the method comprises absorbing water from the process fluid into the hydrogel.

In an embodiment, the method comprises releasing at least one of an electrolyte, a buffer, a mineral, a vitamin, or anti-coagulant from the hydrogel to the process fluid.

In an embodiment, the method comprises moving process fluid through a hollow fiber membrane, the process fluid in indirect communication with the hydrogel through the hollow fiber membrane.

In an embodiment, the method comprises vibrating the hydrogel.

In an embodiment, the method comprises cooling the hydrogel to create a temperature gradient along a length of the hydrogel.

Embodiments may include combinations of the above features.

In a further aspect, the disclosure describes a use of a hydrogel in a renal therapy system, the hydrogel comprising an interpenetrating network of polymer chains, the monomers of the polymer chains having hydrophilic functional groups.

In an embodiment, the monomers comprise at least one of polyacrylamide, acrylic acid, alginate, or chitosan.

In an embodiment, the hydrogel is formed as a plurality of beads having a specific surface area of at least 0.1 m²/m³.

In an embodiment, the hydrogel is cast around hollow filtration fibers.

In an embodiment, the hollow filtration fibres have an inner surface area of between 0.1 to 1.0 m²/m³.

In an embodiment, the hydrogel is a colloidal gel in which water is the dispersion medium.

In an embodiment, the polymer chains are functionalized with chemicals or biological elements to promote sorption of water and toxins in the hydrogel.

Embodiments may include combinations of the above features.

In a further aspect, the disclosure describes a system for controlling a renal therapy device is provided. The system comprises: a controller comprising a non-transitory computer readable storage medium having program code stored thereon, the program code executable by a computer, processor or logic circuit to perform a method comprising: receiving a first power level of a first battery to determine if the first power level of the first battery is above a threshold value; receiving a second power level of a second battery to determine if the second power level of the second battery is above the threshold value; when the first power level is above the threshold value, causing the renal therapy device to be powered by the first battery; when the first power level is below the threshold value, and the second power level is above the threshold value, causing the renal therapy device to be powered by the second battery; wherein the first battery is electrically isolated from the second battery.

In an embodiment, the renal therapy device is a portable wearable renal therapy device.

In an embodiment, the threshold value is the minimum power required to operate the renal therapy system.

In an embodiment, the system comprises sending a signal to an alarm when at least one of the first power level and second power level is below the threshold value.

In an embodiment, the system signaling a clamp to stop process fluid from the renal therapy device when the first power level and second power level are below the threshold value.

In an embodiment, the renal therapy device comprises a sorbent cartridge, the sorbent cartridge comprising a hydrogel configured to absorb or adsorb a toxin from process fluid without use of a dialysate to purify the process fluid. In an embodiment, the system comprises sending a signal to a vibration element to vibrate the sorption cartridge and hydrogel. In an embodiment the system comprises sending a signal to a cooling element to create a temperature gradient across the hydrogel of the sorption cartridge.

In an embodiment, the system comprises signaling a heating element to maintain process fluid at a desired temperature. In an embodiment, the desired temperature is about 37° C.

In an embodiment, the system comprises temperature sensors at opposing ends of the hydrogel to measure a temperature gradient across the hydrogel.

In an embodiment, the system comprises a filtration circuit having an inlet for receiving process fluid and an outlet to return treated process fluid to a patient, the filtration circuit comprising at least one of a process fluid pump; a hemofilter, and an air removal filter; and at least one of a pressure sensor on the filtration circuit, an ultrasonic air detector on the filtration circuit, a blood leak detector coupled to the hemofilter, and an air sensor coupled to the air removal filter. In an embodiment, the system comprises signaling an alarm if an alarm condition is detected by the pressure sensor on the filtration circuit, the ultrasonic air detector on the filtration circuit, the blood leak detector coupled to the hemofilter, and the air sensor coupled to the air removal filter. In an embodiment, the system comprises closing a clamp on the filtration circuit or stopping the process fluid pump if an alarm condition is detected by the pressure sensor on the filtration circuit, the ultrasonic air detector on the filtration circuit, or the blood leak detector coupled to the hemofilter.

In an embodiment, the system comprises communicating with a data system for storing treatment data of a renal therapy device. In an embodiment, the system comprises a sensor for monitoring a pump rotation status, and storing the pump rotation status in the data system.

Embodiments may include combinations of the above features.

In a further aspect, the disclosure describes a method for controlling a renal therapy device. The method comprises: receiving a first power level of a first battery to determine if the first power level of the first battery is above a threshold value; receiving a second power level of a second battery to determine if the second power level of the second battery is above the threshold value; when the first power level is above the threshold value, causing the renal therapy device to be powered by the first battery; when the first power level is below the threshold value, and the second power level is above the threshold value, causing the renal therapy device to be powered by the second battery; wherein the first battery is electrically isolated from the second battery.

In an embodiment, the renal therapy device is a portable wearable renal therapy device.

In an embodiment, the threshold value is the minimum power required to operate the renal therapy system.

In an embodiment, the comprises sending a signal to an alarm when at least one of the first power level and second power level is below the threshold value.

In an embodiment, the first power level of the first battery, and the first power level of the first battery, are received from wireless communication with at least one sensor coupled to the first battery and the second battery.

Embodiments may include combinations of the above features.

Further details of these and other aspects of the subject matter of this application will be apparent from the detailed description included below and the drawings.

### BRIEF DESCRIPTION OF THE DRAWINGS

Reference is now made to the accompanying drawings, in which:

FIG. 1 shows an exploded view of an example sorbent cartridge;

FIG. 2 is an example implementation of a sorbent cartridge in a hemoperfusion system;

FIG. 3 is an example implementation of a sorbent cartridge in a hemofiltration system;

FIG. 4 is an example implementation of a sorbent cartridge in a hemodialysis system;

FIG. 5 is an example implementation of a sorbent cartridge in a peritoneal dialysis system;

FIG. 6 shows a portion of an example sorption cartridge at the interface of first compartment and second compartment of the sorption cartridge;

FIG. 7 shows an example experimental setup to test an example sorbent cartridge; and

FIG. 8A shows perspective view of an example sorbent cartridge according.

FIG. 8B shows a cross-sectional view along the line A-A of FIG. 8A of an example sorbent cartridge having a hollow fiber membrane. FIG. 8C shows a cross-sectional view along the line A-A of FIG. 8A of an example sorbent cartridge having a generally flat or corrugated membrane.

FIG. 9 shows an example controller for a renal therapy device.

FIG. 10 shows an example system for controlling a renal therapy device.

### DETAILED DESCRIPTION OF THE INVENTION

The disclosure herein describes systems and methods of using hydrogel as a sorbent to act as a detoxifier of blood in communication directly or indirectly through a membrane as in hemoperfusion or to clear toxins from ultrafiltrate fluids and dialysate fluids used in hemofiltration, hemodialysis or peritoneal dialysis so that these fluids can be regenerated and reused.

Although terms such as “maximize”, “minimize” and “optimize” may be used in the present disclosure, it should be understood that such term may be used to refer to improvements, tuning and refinements which may not be strictly limited to maximal, minimal or optimal.

The term “connected” or “coupled to” may include both direct coupling (in which two elements that are coupled to each other contact each other) and indirect coupling (in which at least one additional element is located between the two elements).

The term “substantially” as used herein may be applied to modify any quantitative representation which could permissibly vary without resulting in a change in the basic function to which it is related. For example, a drive shaft having a circular transverse cross-section may permissibly have a somewhat non-circular cross-section within the scope of the invention if its rotational driving capability is not materially altered.

The term “sorption” as used herein, refers to both adsorption and absorption. Adsorption is a process that occurs when a gas or liquid or solute (called adsorbate) accumulates on the surface of a solid or more rarely a liquid (adsorbent), forming a molecular or atomic film (adsorbate). It is different from absorption, where a substance diffuses into a liquid or solid to form a “solution”. The term sorption encompasses both processes, while desorption is the reverse process.

The term “small-sized molecules”, as used herein, refers to molecules with a molecular weight lower than 500 Da, such as uric acid, urea, guanidine, ADMA, creatinine.

The term “middle-sized molecules”, as used herein, refers to molecules with a molecular weight between 500 Da and 5000 Da, such as end products from peptides and lipids, amines, amino acids, protein-bound compounds, cytokines, leptins, microglobulins, and some hormones.

The term “ionic solutes”, as used herein, refers to components such as phosphates, sulphates, carbon hydrates, chlorides, ammonia, potassium, calcium, sodium.

The term “process fluid”, as used herein, refers to dialysate fluid, blood, or blood plasma.

“Nano-sized” as used herein, refers to a size of approximately 1-1000 nm, more preferably 1-100 nm.

“Electrolytes” are substances that produce an electrically conductive solution when dissolved in, for example, water, by separation into positive and negative ions. For example, Sodium Chloride (salt) separates into sodium ions and chloride ions. Other electrolytes are bicarbonate, potassium, and phosphate.

“Buffer” solutions are those which resist changes in pH from additions of acidic or basic substances. The resistance to pH change is conferred by an equilibrium between a weak acid and its' conjugate base. Bicarbonate is an example of a buffering substance. Bicarbonate is in 2 equilibrium reactions with carbonate ion and carbonic acid.

The implementation of a wearable renal therapy device that combines continuous or daily blood purification while maintaining a high efficiency for removal of uremic toxins may provide an improvement in the treatment of patients with renal disease, including end-stage renal disease.

While the more continuous and efficient removal of uremic solutes, as well as water and control of electrolytes, is a great advantage of wearable devices, one of the major challenges is the removal of urea. A relatively large amount of urea of up to 24 g needs to be removed daily. Urea has been shown to be difficult to remove with existing sorption methods. Existing wearable renal therapy devices based on sorbent and enzyme technology to allow for the regeneration of dialysis fluid have been used in some prototypes, but they have had some issues with safety, control, size, weight, and cost of expendable components. Electro-oxidation methods have also been used. The problem with electro-oxidation is with the oxidation of chloride leading to the formation of reactive chlorine species, such as chloramines.

Wearable devices utilizing effective sorbent systems can also be used to enhance the efficacy of PD by continuously regenerating the peritoneal dialysate in order to maintain a larger plasma-dialysate concentration gradient. This reduces the amount of time spent by the patient doing exchanges, while still improving toxin clearance. Reduced exposure of the PD catheter to the environment in such a wearable PD device could also prolong the PD technique survival by reducing the risk of bacterial contamination, and so, lower the risk of peritonitis. Continuous glucose infusion by a wearable PD device can reduce functional deterioration of the peritoneal membrane by reducing somewhat the peak level of glucose concentrations that are needed for osmotic fluid removal in conventional intermittent infusion PD. Further, providing portable dialysis devices or artificial kidneys allow patients to engage in normal daily activities while receiving an extended length blood purification treatment without having frequent interruptions or limitations to what they may do.

Portable dialysis devices may be enabled by a system that is able to clear most of the toxins from the blood using no dialysate or as little dialysate as possible. The features of using no dialysate or as little dialysate as possible may require that a substance or substances be used that have an ability to absorb and retain the toxins that need to be removed as well as controlling the electrolyte levels and restoring the buffer solution. There have been other attempts in the past to find such a material. It has been found that activated carbon is able to efficiently remove most of organic uremic organic toxins, middle MW molecules, uric acid, creatinine, and heavy metals with the notable exception of efficient removal of urea. Activated carbon also has minimal effects on electrolyte levels and has no ability to modify or restore buffer levels. Other methods to regenerate dialysate for reuse have used urease enzymes to convert the urea in the fluid to ammonium carbonate which was then removed using zirconium compounds. These zirconium compounds also convert the ammonium carbonate to bicarbonate and remove electrolytes. Electrolyte and buffer levels are then restored to the desired level before being passed through the dialyser again with a calibrated infusion of an electrolyte containing fluid. This process (known as the REDY system) is effective and was used from 1973 to 1993 in a recirculating home hemodialysis system using 6 liters of dialysate, proving that a sorbent-based system can provide adequate therapy. This process is no longer used for home hemodialysis due mainly to higher total treatment costs than single pass systems that use larger volumes of water. There were also lingering concerns of possible negative impacts to patients if the system ever failed to convert all of the ammonia or if some of the chemicals in the zirconium compounds leached into the dialysate if the conversion capacity of these compounds was ever exceeded. This urease, plus zirconium compound and activated carbon system has also been used in trials for a wearable hemodialysis system. A wearable system requires that the dialysate volume be much smaller. This smaller volume of dialysate also makes it more difficult to remove any bubbles that may contain ammonia in the fluid. The total size of the multiple sorbent cartridges required make for a heavier and bulkier system and the high cost of the constituent materials may inhibit widespread adoption.

Hydrogel sorbent (also referred to herein as “hydrogel”) may comprise material with an ability to adsorb large quantities of urea that can also be infused with electrolytes and buffers so that when it is used in a wearable or portable renal therapy system, it can provide all of the sorbent capacity, electrolyte management, and buffer replacement functions required. When hydrogel is used as a sorbent to regenerate dialysate for hemodialysis or peritoneal dialysis, the dialysate volume can be kept very low. Hydrogel sorbent can also adsorb toxins and modify electrolyte and buffer concentrations directly in contact with dialysate or indirectly across a membrane without the use of dialysate to purify hemofiltrate for reinfusion in a hemofiltration system. Hydrogel can also adsorb toxins and modify electrolyte and buffer concentrations of blood directly across a membrane without the use of dialysate in a hemoperfusion system. Hydrogel can be manufactured from low-cost common materials and can be made in such a form that no toxic materials can leach from it.

Hydrogel has been used in other biomedical applications to absorb exudate from wounds, slow release of drugs and other compounds, and as a structural material. Hydrogel has been used in industrial applications to adsorb nitrates, phosphorus, and metals from wastewater. Hydrogel has been used in agriculture to absorb and release water as well as fertilizers such as urea, phosphorus, and other electrolytes into the soil. Hydrogel has not been used as a sorbent for urea and other toxins or to modify electrolytes and buffer levels in biomedical applications.

The hydrogel material can be cast in place directly on a membrane structure or be in a separate chamber in which dialysing fluids are pumped through in order to regenerate the dialysate or reinfusion fluid. The hydrogel may also be in the form of smaller spheres or chopped in order to reduce the flow restriction through the chamber and increase the exposed surface area of the hydrogel material.

In an aspect, an artificial kidney consists of extracting urea and other molecules from blood by dialysis and regeneration of the dialysate fluid when the latter is to be recycled into the dialyser. Regeneration is accomplished by means of adsorbent cartridges, previously this has been enabled by incorporating activated carbon. Activated carbon has the capability to adsorb many uremic toxins including urea. U.S. Pat. No. 3,463,728, which is hereby incorporated herein by reference in its entirety, describes a method of using an activated carbon slurry to augment the capacity of dialysate in a recirculating dialysate system. Activated carbon, however, is not an efficient adsorber of urea and to adsorb the amount of urea for effective clearance in hemodialysis would require upwards of 20 kg of activated carbon per day. A specific method of using urease to clear the dialysate of urea in addition to using carbon and zeolites to manage other electrolytes is described in U.S. Pat. No. 4,581,141, which is hereby incorporated herein by reference in its entirety. Further improvements to these methods are described in U.S. Patent Publication No. 2010/0078387, which is hereby incorporated herein by reference in its entirety, utilizing Zirconium phosphate (ZrP) particles and hydrous zirconium oxide (HZO) particles to also help in managing bicarbonate levels. Urease is expensive and has some issues with the risk of passage of ammonia and bubbles generated in the dialysing fluid. U.S. Pat. No. 9,682,184, which is hereby incorporated herein by reference in its entirety, describes a sorbent cartridge using non-enzymatic urea-binding materials in place of urease. U.S. Patent Publication No. 20110171713, which is hereby incorporated herein by reference in its entirety, describes another sorbent comprising a layer of immobilized uremic toxin-treating enzyme particles intermixed with cation exchange particles. European Patent Publication No. EP1935441A1 (Published on Jun. 25, 2008), which is hereby incorporated herein by reference in its entirety, describes yet another alternative sorbing material utilizing a smectite, nanoclay, a layered double hydroxide, and a modified biopolymer.

In an aspect, the principle of the artificial kidney may be based on ultrafiltration or hemofiltration of the plasma portion of the blood. During hemofiltration, the patient's blood is passed through a set of tubing (a filtration circuit) via a machine to a semipermeable membrane (the filter) where waste products and water are removed. Replacement fluid is added and the blood is returned to the patient. In a similar fashion to dialysis, hemofiltration involves the movement of solutes across a semipermeable membrane. However, the membrane used in hemofiltration is more porous to fluid than that used in most hemodialysis treatments, and no dialysate is used, instead, a positive hydrostatic pressure drives water and solutes across the filter membrane where they are drained away as filtrate. An isotonic replacement fluid is added to the resultant filtered blood to replace fluid volume and valuable electrolytes. This blood and replacement fluid is then returned to the patient. Thus, in the case of recycling fluid for replacement in hemofiltration, a key aspect resides in separating the urea and other toxins from the other components in the ultrafiltrate such as salts which have also passed through the membrane but which must be re-incorporated into the blood in order to maintain the electrolyte composition thereof substantially constant. U.S. Pat. No. 5,211,850, which is hereby incorporated herein by reference in its entirety, describes such a sorbent system to purify plasma ultrafiltered from blood so that it can be returned to the replacement solution. A combination of the two systems described above has also been proposed. U.S. Pat. No. 8,029,454, which is hereby incorporated herein by reference in its entirety, describes such a hemodiafiltration system using sorbents for fluid regeneration for both the hemodialysis aspect and the hemofiltration aspect.

Direct hemoperfusion systems, or systems that perform no ultrafiltration, yet which adsorb toxic substances directly from the blood have also been proposed. U.S. Pat. No. 4,169,051, which is hereby incorporated herein by reference in its entirety, describes carbon sorbent spheres coated with a membrane material in order to reduce coagulation of the blood it is in contact. Others have used different adsorbent materials coated in a membrane. In general, hemoperfusion systems are not widely used for artificial kidney systems due to higher costs and the inherent lower efficiency of urea removal. In general hemoperfusion systems target specific toxins not generally removed well by regular hemodialysis or hemofiltration. These are usually meant not for renal replacement therapies but as an adjunct to another renal replacement therapy in order to improve the clearance of targeted molecules. U.S. Pat. No. 6,878,269, which is hereby incorporated herein by reference in its entirety, describes a sorbent column containing spherical hydrogel particles of a cellulose acetate used to remove β2-microglobulin and chemokines. As noted above, the adsorbent for regenerating the dialysate or ultrafiltrate is usually activated carbon. However other adsorbents have been proposed for the removal of substances from dialysis fluids or ultrafiltrate. U.S. Pat. No. 3,874,907, which is hereby incorporated herein by reference in its entirety, describes microcapsules consisting of a crosslinked polymer containing sulphonic acid groups and coated with a polymer containing quaternary ammonium groups, for use in regenerating the dialysate. Examples of the sulphonated polymer include sulphonated styrene/divinyl benzene copolymer and examples of the coating polymer include those obtained by polymerization of for instance vinyldimethylamine monomers.

The disclosures noted above are directed to both dialysing, ultrafiltration and hemoperfusion devices, wherein various substances may be used as sorbents. The disclosures also includes patents granted for the use of specific sorbent materials for use in renal therapy devices. However, a problem with the systems of the disclosures above, that they are still too large due to limited sorption capacity of the materials, have the risk of eluting toxic chemicals such as ammonia or chlorine or not efficient, or all of the above in order to allow small, desktop-sized or wearable dialysing and ultrafiltration systems. The raw materials cost for these systems is also high. This limits their ability to be a lower cost solution to the expensive existing methods of renal therapy.

An object of the present invention is to overcome the problems associated with the devices of the prior art and to provide a compact and efficient sorption system for use in hemodialysis and peritoneal dialysis systems portable renal therapy systems and in a wearable renal therapy system.

Aspects of various embodiments are described through reference to the drawings.

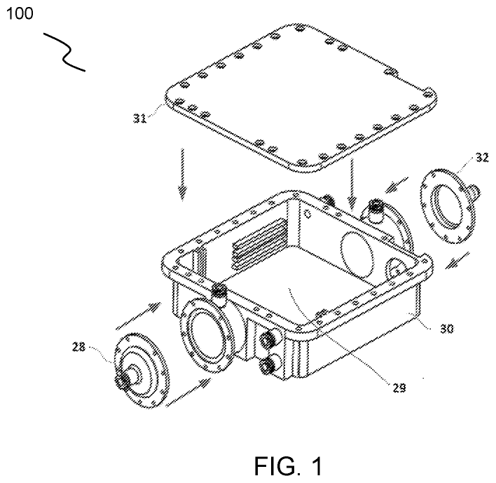

FIG. 1 illustrates an example sorption cartridge (100). In an embodiment, the sorption cartridge 100 is provided for the removal of water and/or waste materials such as toxic substances from hemodialysis, hemofiltration and peritoneal fluids, allowing little or no dialysate volume and thereby allowing a small, desk-top size or wearable hemodialysis, hemofiltration or peritoneal dialysis system. The sorption cartridge of the present disclosure may take the form of a cartridge, comprising a rigid or flexible housing (30) comprising the sorbing materials, e.g. hydrogel sorbent. The inlet port (32) and outlet port (28) may be removable so as to allow for the optional addition of a membrane in order to separate the blood pathway from the hydrogel sorbent in the reservoir (29). Reservoir (29) may also be divided into one or more compartment which are described below. The sorption cartridge (100) may comprise an absorbing, adsorption, and/or ion-exchange material composed of a hydrogel sorbent. The hydrogel material may adsorb or absorb, or adsorb and absorb water, small-molecules such as uremic toxins, medium-molecules, and may also control electrolyte and buffer levels in the process fluid (e.g. blood, plasma or dialysis fluid). The hydrogel sorbent may be cast in place, e.g. within reservoir (29), or it may comprise beads of hydrogel. The cartridge lid (31) may be removable from the cartridge housing (30) in order to allow the placement of the hydrogel sorbent material and optional membrane, heating elements, and sensors. In an embodiment, the sorption cartridge may have dimensions of 10 cm×10 cm×3 cm. The reservoir may comprise approximately 300 mL of hydrogel (about 300 g weight) and the total weight of the sorption cartridge, including the container, may be between about 500 and 700 grams.

Sorption cartridge 100 may comprise a membrane configured to remove water and waste material (e.g. small-molecules and medium-molecules) from a process fluid. The membrane may be shaped as a hollow fiber, generally flat, generally corrugated, or other suitable shape to separate water and waste material from process fluid. The membrane may define a flow path through the sorbent cartridge, e.g. a hollow fiber membrane may be configured to convey process fluid through the cartridge which may include defining a flow path through the hydrogel within the cartridge. Example flat membranes may include Spectrum™ Spectra/Por™ 1-4 Standard RC Dialysis Membrane in Flat Sheets, having manufacturing nos. SML132677, SML132686, SML132723, SML132712 respectively. Examples of hollow fiber dialysis membranes include Elisio™_H membrane (e.g. models: ELISIOV11H or ELISIOV15H) provided by Nipro Corporation, Polynephron™ membrane provided by Nipro Corporation, Asymmetric Tri-Acetate (ATA) Membrane provided by Nipro Corporation, Membrana™ Purema™ H capillary membrane provided by 3M Company, and Membrana™ Diapes™ capillary membrane provided by 3M Company. In an embodiment, the hollow fibers of the hollow fiber membrane have a thickness of less than 0.5 mm. In another embodiment, the hollow fibers of the hollow fiber membrane have a thickness of less than 200 microns.

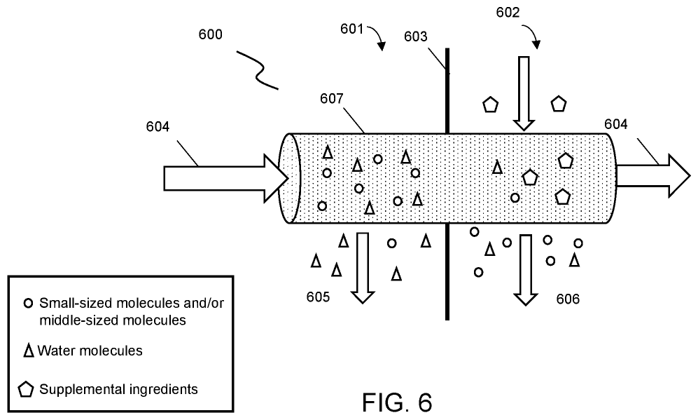

In an embodiment, a sorption cartridge according the disclosure herein may comprise one more compartments. In an example, a sorption cartridge may have two compartments. A first compartment may be configured for water removal and second compartment may be configured for removal of middle-sized molecules and/or small-sized molecules such as uremic toxins. The first compartment configured for water removal may comprise a membrane, as described above, that may remove water and other solutes (e.g. waste material) from a process fluid by ultrafiltration across the membrane to an ultrafiltrate. The second compartment configured for toxin removal (e.g. urea) may comprise the membrane, described above, which may be embedded in hydrogel sorbent. FIG. 6 illustrates a portion 600 of a sorption cartridge, according to the disclosure herein, at the interface of first compartment 601 and second compartment 602. As shown, first and second compartments 601, 602 are connected in series such that process fluid 604 flows from one compartment to another through membrane 607. The interface 603 between the first compartment 601 and second compartment 602 may be defined by a wall, permeable barrier, or allow direct contact between compartments 601, 602. The portion of membrane 607 in the first compartment 601 may separate water molecules, middle-sized molecules, and/or small-sized molecules by ultrafiltration where the ultrafiltrate 605 may be pumped away. In an example, ultrafiltrate 605 may comprise dialysate. Compartment 602 may comprise hydrogel sorbent, according the disclosure herein, which interfaces with membrane 607 at a membrane-hydrogel interface. As shown in FIG. 6, membrane 607 in compartment 602 is embedded in hydrogel 606 such that the exterior surface of the membrane 607 interfaces with hydrogel 606. Small-sized molecules, including toxins such as urea, may be absorbed water within hydrogel 606 and/or adsorbed onto the hydrogel. The arrangement of the compartments is not limited to the illustrated embodiment and may be reversed. Similarly, the illustrated embodiment shows membrane 607 as a hollow fiber membrane embedded in hydrogel, the hydrogel surrounding the membrane; however, other types and shapes of membranes may be used. In an example, the first compartment 601 may be configured as a hemofilter and the second compartment 602 may comprise hydrogel such that each compartment is defined in a single housing having unitary structure. Continuing the example, the portion of the fibers in the first compartment that are not covered with hydrogel sorbent may be used to provide needed ultrafiltration and fluid removal which is pumped away from the cartridge to another container. In another example, a sorption cartridge according the disclosure herein, may only one compartment comprising the elements of compartment 602 illustrated in FIG. 6.

In an embodiment, hydrogel according to the disclosure herein, may be configured to release supplemental ingredients such as electrolytes, buffer, minerals, vitamins, and/or other substances to the process fluid. For example, hydrogel may also comprise sodium bicarbonate for bicarbonate ion control and/or anti-coagulant (e.g. heparin or citrate) to aid in anti-coagulation, each of which may be released from the hydrogel to the process fluid. The hydrogel of the sorbent cartridge may be pre-loaded with supplemental ingredients such that the supplemental ingredients are desorbed to the process fluid when in use.

In an embodiment, the membrane-hydrogel interface may be functionalized by to promote water and/or toxins to move from the process fluid across the membrane to absorb into, or adsorb onto, the hydrogel. In an example, the molecular structure of the monomers of the polymer chains, which make up the polymeric structure of the hydrogel, may have hydrophilic functional groups that confer hydrophilicity to the hydrogel to promote absorption of water by the hydrogel through the membrane. In another example, chemicals and/or biological elements may be added to the hydrogel to attract toxins in the process fluid. The membrane may also be modified by chemicals to promote toxins to cross the membrane-hydrogel interface into the hydrogel.

Hydrogels described herein, may be cast onto a membrane, and/or may have a membrane embed within the hydrogel, such that the hydrogel has a layer having a thickness. The thickness of the hydrogel may be configured to provide a concentration gradient to absorb toxins from process fluid. In an embodiment, the hydrogel layer has a thickness of is greater than or equal to about 1 mm. In another embodiment, the hydrogel layer has a thickness between 1 mm and 3 mm. In another embodiment, the hydrogel layer has a thickness of greater than or equal to 3 mm.

In another embodiment, reservoir (29) of sorption cartridge 100 may comprise hydrogel sorbent in the form of a plurality of hydrogel beads. Process fluid, e.g. blood, plasma, or dialysate fluid may be configured to be direct contact with the bead shaped hydrogel sorbent as it flows through the sorbent cartridge. In an example, each hydrogel bead may have a diameter of greater than or equal to about 1 mm. In another embodiment, the hydrogel beads each have a diameter between 1 mm and 3 mm. In another embodiment, the hydrogel beads have a diameter of 3 mm to 10 mm.

The sorption cartridge of the present disclosure is distinguished from the prior art devices in that it makes use of a hydrogel with a high capacity to sorb urea in order to allow small dimensions for wearability. The sorption system (the ability of the hydrogel to adsorb and/or absorb), and optional release system (the ability of the hydrogel to release electrolytes, buffer, minerals, vitamins or other substances to the blood, plasma or dialysis fluid), of the sorption cartridge(s) described herein may have a temporary use until it reaches its maximum sorption capacity. The content of the hydrogel sorbent can be customized to the individual patient needs. A sorbent cartridge according to the present disclosure may form a disposable and replacement part of the renal therapy system, and can be replaced by a fresh sorbent cartridge, for instance when it has been saturated with toxic substances, or if one or more of the components supplemented to the plasma have run out.

The sorption cartridge of the present disclosure may be used for filtering or purification of the blood of patients with a (developing) renal failure. In an embodiment, the sorption cartridge may be used in a wearable artificial kidney device, but can also be embodied in desktop sized equipment or in adapted hemodialysis or peritoneal dialysis equipment.

The sorption cartridge of the present disclosure may be combined with suitable equipment to expose it to toxins in the blood in order to adsorb those toxins is able to perform some of the functions which normally will be done by a properly functioning human or animal kidney, in particular, filtering of blood and regulation and control of the content of substances in the blood. The sorption cartridge of the present disclosure comprise a sorption system for capturing toxic substances from the blood and optionally a release system for releasing minerals, vitamins or other substances to the blood, a filter for separating blood cells from blood plasma on the basis of hemofilter.

A sorption cartridge according to the present disclosure may be configured to remove urea and other toxic material from blood, plasma or dialysis fluid. Although urea is only toxic in the body at high concentrations (above 15 g/kg) and is neither acidic nor alkaline when dissolved in water, the body generates large volumes each day as part of protein metabolism (greater than 1800 mg per day), which should be removed or the concentration of urea would build-up. Urea is highly soluble in water having a solubility of in water of about 1079 g/L at 20° C. Urea, being a molecular substance, does not dissociate into ions but will solvate with water by forming hydrogen bonds which may occur in two ways: hydrogen atoms bonded to water will align to the partially electronegative area of the amine group; and/or oxygen of the carbonyl and the hydrogen bonded to the amine group may be attracted and associated to the oxygen end of a water molecule.

Because urea is soluble in water (solubility of ˜1000 g/L depending upon the temperature) allows urea to be readily diffused across a membrane in standard hemodialysis. However, standard hemodialysis is unable to take advantage of the ability of water to dissolve very high concentrations of urea because the concentration of urea in the dialysate must always be kept lower than the concentration of urea in the blood in order to maintain a concentration gradient for removal of urea from the blood.

A hydrogel sorbent comprises a three-dimensional network of cross-linked polymer chains. The hydrogel sorbent may have a high water content, and may be able to swell and shrink as it absorbs or releases water. A hydrogel sorbent may comprise of a network of polymer chains that may be hydrophilic. The molecular structure of the monomers of polymer chains, which make up the polymeric structure of the hydrogel, may have hydrophilic functional groups that confer hydrophilicity to the hydrogel. The ability of a hydrogel to expand/swell is a function of the density and cross-linking of the gel. In an embodiment, the hydrogel sorbent may be a colloidal gel in which water is the dispersion medium. In another embodiment, a hydrogel sorbent may comprise a three-dimensional solid resulting from hydrophilic polymer chains being held together by cross-links. Because of the inherent cross-links, the structural integrity of the hydrogel network does not dissolve from the high concentration of water. As a result of the high water concentration, hydrogels are able to absorb large quantities of water-soluble substances such as urea. Hydrogels can also provide absorption by adsorbing electrolytes and uremic toxins into the gel matrix pore structure so that the concentration of the fluids in contact with the membranes that also contact the blood can be kept at a lower concentration than in the blood and so maintain a concentration gradient that will continue to clear urea from the blood. The reaction for toxins to adsorb onto the hydrogel polymeric structure may comprise physisorption, which is a physical entrapment of the toxin molecules within the solid pore structure. The sorbing materials may be functionalized, such as to exhibit improved sorbing properties of toxic substances such as urea as compared to the non-functionalized material. In an embodiment, the sorbing materials are hydrogels with a high water absorbing capacity and an interpenetrating network of pores in order to create a large specific surface area. The hydrogel may enable very high sorption efficiency and therefore enable a small-sized, lightweight and wearable device.

Hydrogels can be prepared in different ways from various materials. Examples of suitable hydrogel materials include polyacrylamide, acrylic acid such as polyacrylic acid, alginate, and chitosan. To enhance the available surface area for adsorption hydrogels may have an interpenetrating network. Increasing surface area may increase the rate and capacity of toxin removal. Hydrogel may be formed as small hydrogel beads, which, in an embodiment, have the specific surface area of at least 0.1 m²/m³. As described herein, hydrogel may be disposed to surround hollow filtration fibres, such as those used in ultrafiltration. In an embodiment, the hollow fibre inner surface area is between 0.1 to 1.0 m²/m³.

Other hydrogel properties that influence toxin removal and/or water removal include pore size, water capacity, and monomer concentration.

The following are examples of methods of manufacturing a hydrogel:

### Synthesis of Simple Poly-Acrylamide (PAAm) Hydrogel:

a. To synthesize poly-acrylamide hydrogel 2 g (28.1 mmol) of Acrylamide (AAm) and 100 mg (0.65 mmol) MBAAm are mixed in a dry 50 mL reaction flask. The concentration of AAm and MBAA can be varied based on the desired porosity and water absorption capacity needed with the hydrogel. The formula can be maximized or minimized and the ratio of AAm and MBAAm can be varied to obtain hydrogels with varying crosslinking and also water absorption capacity.
b. To the above flask add 20 mL of deionized water and gently swirl the reaction flask using a magnetic stir bar on a magnetic stir plate until both the reactants are completely dissolved.
c. The resultant solution is deoxygenated for 15 minutes to prevent the reaction between the oxygen and the initiators.
d. Now add 50 μL of Ammonium persulfate (APS) solution 10% w/v and 10 μL of TEMED into the reaction flask to initiate the polymerization. The amount of initiator can be varied to modify gelation time.
e. Swirl the reactor flask five to six times by hand and then pour the resultant mixed solution into an appropriate dish under nitrogen.
f. The poured solution is left under room temperature for 2 hours to polymerize and form the hydrogel.
g. The resultant hydrogel is now immersed into deionized water for up to 2 days with water changes three times a day to remove any unreacted monomers.
h. After the cleaning process, the resultant hydrogel is transferred to an appropriate container for further processing. This procedure may yield a simple hydrogel without any functionalization.

### Synthesis of PEG-Functionalized Poly-Acrylamide (PAAm-PEG) Hydrogel:

a. To synthesize PEG-functionalized PAAm hydrogels, APS (0.056M) and TEMED (0.32M) are used as redox initiator system.
b. AAm (1.0 g) APS (1 mL) and MBAAm (0.05 g) were added to 50 mL reactor flask and 5 mL of distilled water is added to it.
c. Then PEG (concentrations between 4.8-20 wt %) was dissolved in the monomer solution, the solution is purged with nitrogen gas for 10 minutes to remove any oxygen present that can react with the initiator.
d. To the above solution add 0.2 mL of TEMED and under a nitrogen environment, transfer the solution into a polypropylene Petri dish.
e. Based on the targeted pore size to be formed on the hydrogel, PEG wt. % and the PEG molecular weight is changed. PEG can be found in many sizes, ranging from <100,000 Da to >1 Million Da. Hydrogels created according to the present disclosure typically use PEG sizes under 100,000 Da. Other porogens can be used as an alternative to PEG, including various molecular weight Polyvinyl alcohol (PVA). In this example, PEG may comprise 4.8 wt % using PEG 4000 (PEGs generally do not react with any other components of the reaction).
f. Leave the Petri dish for 24 hours for the polymerization to continue by maintaining the temperature between 20-27° C.
g. Upon completion of the reaction, the hydrogels are cut into specified shape and size as required and are placed in a large excess amount of water for at least 72 hours with regular water changes at least three times a day to wash away any excess reagents that are unreacted and the pore forming agent.
h. Samples are then dried under room temperature to the constant weight required or swollen as per requirement using water/buffer solution.

### Formulation of Hydrogel Containing Dialysate Buffer:

a. Preparation of Acidifying agent:
To prepare acidifying agent, follow the formula provided below:

| | | |
|---|---|---|
| Sodium Chloride (NaCl) | 21.48 | g |
| Potassium Chloride (KCl) | 0.65 | g |
| Calcium Chloride (CaCl2•2H2O) | 0.772 | g |
| Magnesium Chloride (MgCl2•6H2O) | 0.53 | g |
| Purified Water | 100 | mL+ |

To the above solution add citric acid at the required concentration to induce the anti-coagulant effect. The concentration of citric acid should be between 0.1-2.5 mEq/L to induce the anticoagulant effect. The addition of citric acid increases the pH markedly. Hence, the pH may be adjusted after the addition of an alkalizing agent.
b. Preparation of Alkalizing Agent:

| | | |
|---|---|---|
| Sodium bicarbonate (NaHCO₃) | 7 | g |
| Purified water | 100 | mL |

Add above quantity of NaHCO₃ to 100 mL of pure water this is added to the above prepared acidifying agent at a weight ratio of 1:1.26:32.74
Then citric acid is added to adjust the pH between 7.25-7.45 (physiological pH) anything below 7.25 causes acidosis and above 7.45 causes alkalosis.
After 2 hours, the obtained hydrogel is now swollen using the dialysate buffer instead of water. The hydrogel is cut into the required shape and then it is placed in the beaker containing dialysate and is swollen up to 72 hrs. The dialysate buffer is changed at least twice a day to replenish the dialysate buffer and to remove any unreacted components leftover after synthesis of the hydrogel.
The buffer mentioned above can also be directly used in place of deionized water in the synthesis of the hydrogel, this will allow the hydrogel to initially form at the pH required for a dialysate. Once the hydrogel is fully swollen, it can be either cut into the required shape to fit into the diffusion chamber and the diffusion of toxin molecules is monitored.

Properties of hydrogel such as their large water absorption capacity and the porosity formed on the hydrogel allows them to have high levels of saturation for the water-soluble compounds. This porosity allows the compounds diffused into the hydrogel through the semi permeable filter to diffuse slowly into deeper layers of hydrogel, therefore creating a layer with less concentration in and around the semi-permeable membrane leading to the continuous influx of the toxin molecules.

The performance of a hydrogel according to the disclosure herein may be measured by removal of urea, creatinine and phosphate. In an example, 34.6 g of hydrogel was prepared and immobilized in a cartridge. Urea was added to 2 liters of porcine blood to simulate uremic levels. Urea was also added to the 300 ml of dialysate to raise its urea concentration to that same level which negated any removal due to equilibrium with dialysate so that all urea reduction will be due solely to sorption by the hydrogel in the cartridge. The blood was dialysed and the dialysate regenerated by passing through the adsorbent hydrogel. Samples of the blood were taken initially and at 15 minutes.

TABLE 1
VetScan VS2 Kidney Profile Plus Data Summary

| | T = 0 | T = 15 |
|---|---|---|
| | min | min |
| BUN mgldl | 180 | 102 |
| Calcium mgldl | 8.3 | 9.3 |
| Creatinine mgldl | 1.1 | 0.4 |
| Phosphate mgldl | 6.4 | 3.3 |
| Sodium mmolll | 147 | 147 |
| Potassium mmolll | 4.9 | 4.6 |
| Chlorides mmolll | 106 | 101 |
| TCO2 mmolll | 27 | 26 |

Analysis of the experiment summarized in Table 1 indicated that (1) electrolytes of sodium, potassium, calcium, chlorides, as well as TCO2, remained relatively stable the entire time; (2) both BUN and creatinine levels dropped significantly (˜40-50%) in 15 minutes; and (3) phosphate levels dropped during the experiment. In an embodiment, an ideal sorbent for a wearable renal therapy device would be able to remove urea, creatinine and to lower phosphate levels without having significant effects on electrolytes, or buffer concentrations. The results of the experiment illustrated in Table 1 indicate all of these effects. Moreover, the experiment demonstrated the urea reduction of 43% in 2.3 liters of fluid in a time of 15 minutes using only 34.6 grams of the sorbent. This may translate to the removal of 3.8 grams of urea for a sorbent removal rate of 110 mg/g (urea/sorbent). At this rate, 24 grams of urea for a daily therapy could be removed using only 216 grams of the hydrogel sorbent. Phosphate and creatinine levels would also be reduced. Creatinine and middle molecules could also be further reduced by using activated carbon. Electrolyte levels could be adjusted by the addition of ion exchangers and replacement as needed.

Diffusion of uremic toxins from the high-temperature area to low-temperature area (Soret effect) may improve the ability of hydrogel to absorb toxins. Using the principles of soret effect in the diffusion of uremic toxins through hydrogel creates an unsaturated area around the semi-permeable membrane and on the top layers of the hydrogel. The differential temperature in the hydrogel compartment keeps one part of the hydrogel at one specific temperature while the other half is at a different temperature. This kind of differential temperature arrangement creates thermophoretic mobility in the molecular compounds in the solution/hydrogel leading to their diffusion/movement from higher temperature area to lower temperature area. The thermodiffusion response of a solute is quantified by the Soret coefficient, S_{T}, which is proportional to the concentration gradient that builds as a response to a thermal gradient. A positive Soret coefficient indicates that the solute accumulates on the cold side (thermophobic), while a negative sign denotes drift towards the warm side (thermophilic). As discussed in D. Niether, S. Di Lecce, F. Bresme and S. Wiegand, Phys. Chem. Chem. Phys., 2017, DOI: 10.1039/C7CP05843H, the disclosure of which is incorporated herein by reference in its entirety, urea solutions are thermophobic for the concentrations of 0.1 M and 0.05 M (which are equivalent of 2 g/L and 1 g/L urea solutions, respectively, which are physiologically relevant for dialysis).

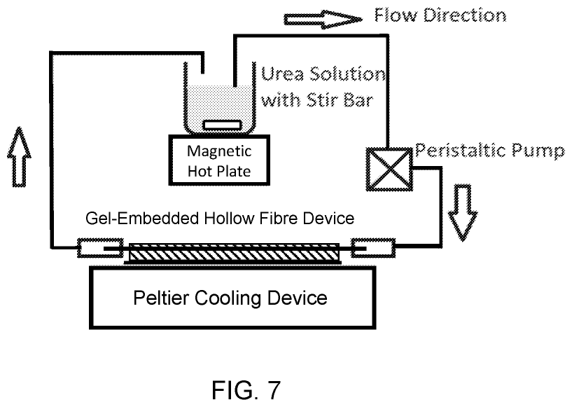

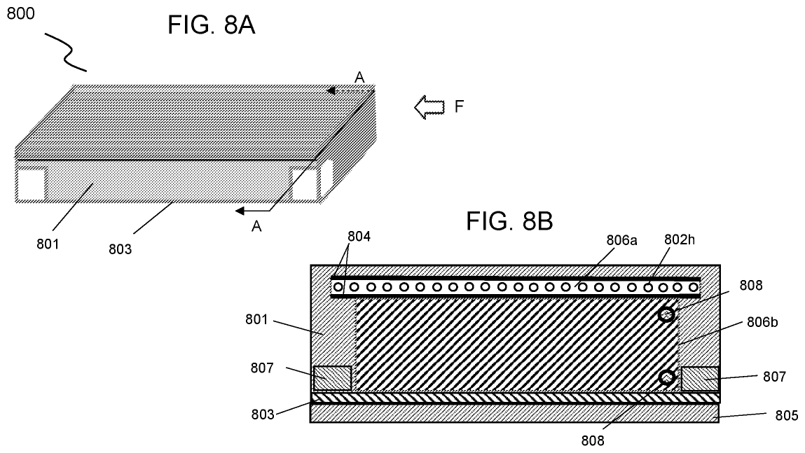

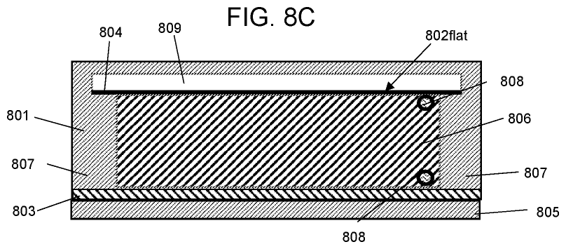

As disclosed herein, the soret effect was found to improve the ability of a hydrogel absorb uremic toxin. In an example, maintaining a temperature gradient of ˜20° C. across a small thickness (3 mm) of hydrogel using a peltier cooling device demonstrated improved removal of urea from a urea solution. If the temperature gradient is maintained, the Soret effect drove diffusion of urea from the fibres towards the cooler part of the gel, and utilization of the Soret effect increased the removal capacity of our gel fibre devices when compared to a control experiment. FIG. 7 illustrates the a setup of the experimental apparatus testing a hydrogel embedded with hollow fibers such as the one illustrated in FIGS. 8A and 8B; however, for the control experiment, the Peltier cooling device was excluded. The experimental procedure was to:
1. Keep the urea solution at a temperature of ˜37° C., and the Peltier device at 17° C. (20° C. temperature gradient);
2. Pump the urea solution through at low flow rates to ensure pressure does not cause leaks to occur (in this case, 17 mL/min was used, resulting in an approximate pressure of 6 psi);
3. Take samples at the following times (in minutes): 0, 5, 10, 15, 20, 25, 30;
4. Record the final volume of urea solution at the end of the experiment; and
5. Analyze each sample for concentration of urea, and determine total removal at each time interval
   Which provided the results in Tables 2 and 3.

TABLE 2
Results from Control Experiment

| | | | Urea | Apparent Urea |
|---|---|---|---|---|
| | | Urea | removed via | removal via |
| Time | [Urea] | in System | ultrafiltration | Diffusion |
| (min) | (mg/mL) | (mg) | (mg, cumulative) | (mg, cumulative) |
| 0 | 2.019 | 302.85 | N/A | N/A |
| 5 | 2.002 | 291.52 | 10.05 | 1.28 |
| 10 | 2.032 | 282.38 | 20.14 | 0.33 |
| 15 | 1.881 | 264.13 | 29.92 | 8.8 |
| 20 | 1.930 | 247.72 | 39.45 | 15.69 |
| 25 | 1.950 | 242.5 | 49.15 | 11.2 |
| 30 | 1.963 | 234.78 | 58.93 | 9.14 |

Starting Volume: 150 mL
Final Volume: 120 mL
Filtration Rate: 1 mL/min

TABLE 3
Results using Soret Effect in Experiment

| | Measured | | Urea | Apparent Urea |
|---|---|---|---|---|
| | [Urea] | Urea | removed via | removal via |
| Time | in sample | in System | ultrafiltration | Diffusion |
| (min) | (mg/mL) | (mg) | (mg, cumulative) | (mg, cumulative) |
| 0 | 2.011 | 201.1 | N/A | N/A |
| 5 | 1.942 | 187.77 | 9.88 | 3.45 |
| 10 | 1.601 | 159.44 | 18.74 | 22.93 |
| 15 | 1.888 | 148.28 | 27.46 | 25.36 |
| 20 | 1.793 | 147.24 | 36.67 | 17.2 |
| 25 | 1.89 | 138.11 | 45.87 | 17.12 |
| 30 | 1.865 | 131.43 | 55.26 | 14.42 |

Starting Volume: 100 mL
Final Volume: 70 mL
Filtration Rate: 1 mL/min

Continuing the example above, during the experiment, over the 30 minute duration, the experiment utilizing the Soret effect was 1.5 times as effective as the control experiment, with mostly the same parameters. The only different parameter was the initial volume, which was greater in the control and normally favors greater diffusion in the control. The results imply that using the Soret effect is at least 1.5 times more effective, if not slightly more.

FIG. 8A illustrates a perspective of an a embodiment of a sorbent cartridge according to the present disclosure, and FIGS. 8B and 8C each illustrate cross-sectional views along the line A-A of FIG. 8A where FIG. 8B illustrates an example sorbent cartridge comprising a hollow fiber membrane, and FIG. 8C illustrates an example sorbent cartridge comprising a generally flat or generally corrugated membrane. As shown in FIG. 8B sorbent cartridge 800 may comprise a housing 801, hydrogel 806a, 806b, and membrane 802h. Housing 801 may be a flexible or ridge material such as acrylic glass. In an non-limiting example, hydrogel 806a and 806b is a hydrogel material formed from polyacrylamide (PAAm). Membrane 802h, which is illustrated as a hollow fiber membrane in FIG. 8B, is defined by hydrogel 806. Optional support member(s) 804, e.g. wire mesh, may provide rigidity to the hydrogel 806a and membrane 802h for positioning within sorbent cartridge 800. Support member 804 is configured to provide fluid communication between hydrogel 806a and 806b allowing solutes in the hydrogel, e.g. uremic toxins, to move between hydrogel 806a and 806b. Conductive member 803 may be positioned a distance, e.g. 3 mm, away from membrane 802h and configured to couple with a cooling element 805. Conductive member 803 may be configured to transfer heat uniformly across the surface interface between hydrogel 806 and conductive member 803. Conductive member 803 may be made of a metallic material or other suitable heat conductive material. Cooling element 805 may be a an integral part of sorbent cartridge 800 or part of a renal therapy system to which sorbent cartridge is coupled. When sorbent cartridge 800 is in use, process fluid enters membrane 802h as shown by flow direction F, which illustrates the flow direction of the process fluid through hydrogel 801. The process fluid may be at about 37° C. (plus/minus 1° C.). A heating element (not shown) may be provided proximate to membrane 802h, e.g. along the surface of housing 801 opposite cooling element 805 and/or conductive member 803, to maintain the process fluid at a desired temperature, e.g. 37° C. Cooling element 805 may create a temperature gradient across the distance of the hydrogel 806a and 806b between conducive member 803 and membrane 802h. Uremic toxins, and other solutes in the process fluid, will move into the hydrogel 806a 806b. When the temperature gradient exists across the hydrogel, the Soret effect, which is discussed above, may improve uremic toxin diffusion into the hydrogel from the process fluid. Sensors 808 may be provided at a proximate end of hydrogel 806b near membrane 802h and distal end of hydrogel 806b near cooling element 805 to measure a temperature gradient across the hydrogel.

FIG. 8C illustrates an example sorbent cartridge having the same functionality and elements as the sorbent cartridge illustrated in FIG. 8B; however, the hollow fiber membrane is replaced with a generally flat or generally corrugated membrane 802. Generally flat or generally corrugated membrane 802 may be positioned directly on hydrogel 806 and optionally held in position by supporting member 804. Flow path 809 may be defined by the membrane and housing 801. As process fluid move through flow path 809, uremic toxins, and other solutes in the process fluid, will move into the hydrogel 806 as described above with reference to FIG. 8B.

As disclosed herein, vibration may increase the rate of sorption of solutes, such as uremic toxins, into a hydrogel. The transfer of uremic toxins, e.g. urea, from a process fluid may be rapid initially as urea adsorbs onto the surface of the hydrogel, then slows as it must penetrate into the pores to be adsorbed by inner surfaces. In an example, vibration was demonstrated to increase the rate of sorption or urea into a hydrogel. Equivalent amounts of urea solution (with the equivalent of 2.5 grams of urea per 1 liter of water) was placed onto two trays of a hydrogel, each having the same formulation, the hydrogel having a surface area of 124 mm by 55 mm (0.007 m² surface area) with the thickness of 10 mm. Four 5 volt 11000 RPM vibration motors (DigiKey number is 1528-1177-ND) were installed on one tray to vibrate at frequency of 183 Hz, while no vibration motors were connected to the control tray. Table 4, below, illustrates the increased removal of urea due to vibration

TABLE 4
Percentage of the urea reduction in the urea solution

| Formulation | F8 Vibration (%) | F8 Control (%) |
|---|---|---|
| Initial | 0.0 | 0.0 |
| 60 Minutes | 11.5 | 11.7 |
| 180 Minutes | 58.2 | 18.3 |

In an embodiment, a vibration element 807 may be provided to vibrate a sorption cartridge according the disclosure herein. As shown in FIG. 8B, sorption cartridge 800 may comprise a vibration element 807 configured to vibration hydrogel 806a and 806b. Vibration element may be external to the sorption cartridge and part of a renal therapy system to which the sorption cartridge is coupled. When in use, the vibration element 807 may vibrate a sorption cartridge, and hydrogel therein, to increase the rate of sorption of solutes, such as uremic toxin, from the process fluid into the hydrogel.

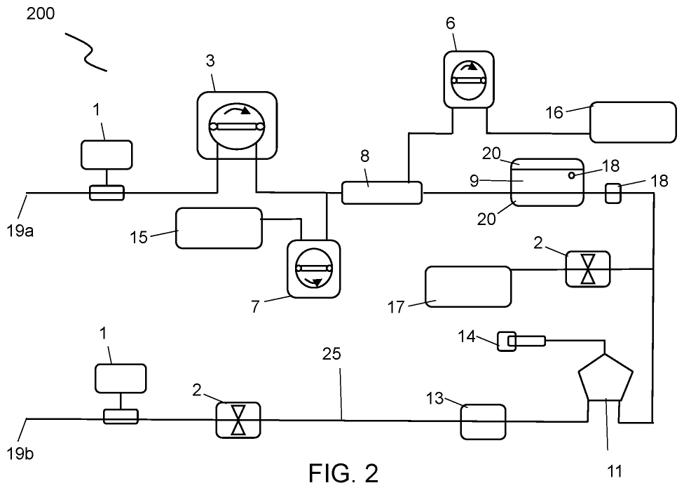

FIGS. 2-5 illustrated example renal therapy fluid systems wherein sorption cartridges according to the present disclosure are incorporated into hemoperfusion, hemofiltration, hemodialysis, or peritoneal dialysis systems. The illustrated example systems do not limit how sorption cartridges according to the present disclosure may be used.

A sorption cartridge according to the present disclosure may be placed in a dialysis fluid system of a hemodialysis or peritoneal dialysis system enabling the removal of toxins consisting of small-sized molecules, middle-sized molecules, and ionic solutes from the dialysis fluid, such as those illustrated in FIGS. 2-5. The sorption cartridge may continuously purify dialysate fluid, keeping the toxin concentration in the dialysis fluid flow, which may improve hemodialysis and peritoneal dialysis efficiency, and may reduce the consumption of dialysis fluid. An additional and optional function of the sorption cartridge is to release ingredients for supplementing of the blood such as calcium, magnesium, anticoagulation agents, antimicrobial agents, other minerals, specific medicaments etc. Supplemental ingredients for delivery can be included in the hydrogel solution, and upon gelation, will be dispersed within the gel matrix of the formed hydrogel. Diffusion of the supplemental ingredients from the hydrogel to the process fluid, e.g. blood or dialysate fluid, occurs due to the concentration gradient of the supplemental ingredients for delivery. This optional delivery of supplemental ingredients may simplify the operation of existing hemodialysis systems and may reduce the chance of occurring peritoneal infections in a peritoneal dialysis system.

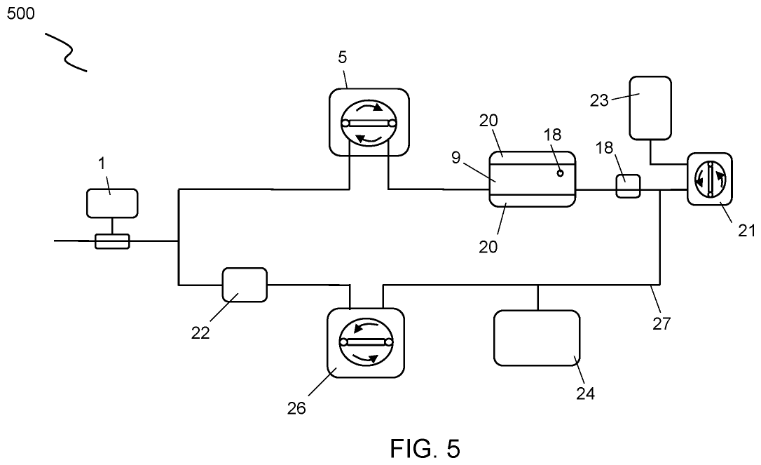

A sorption cartridge according to the present disclosure may also form part of a wearable peritoneal dialysis system wherein the sorption cartridge package is placed in the flow path of a wearable peritoneal dialysis system. Due to the continuous removal of toxins by the sorption cartridge, the volume of dialysate fluid can be reduced. The wearable peritoneal dialysis system, such as the system illustrated in FIG. 5, may comprise a tubular access system to the abdominal cavity and a unit comprising a fluid pump, power, sensors, electronic control, a facility to place and replace said sorption cartridge package, typically on a daily basis and a system to dispose of removed fluid. An additional and optional function of the sorption filter is to release ingredients for supplementing the blood, such as calcium, anticoagulation agents, antimicrobial agents, minerals, specific medicaments etc. This option will enhance the operation of the peritoneal dialysis system and will reduce the chance of occurring infections.

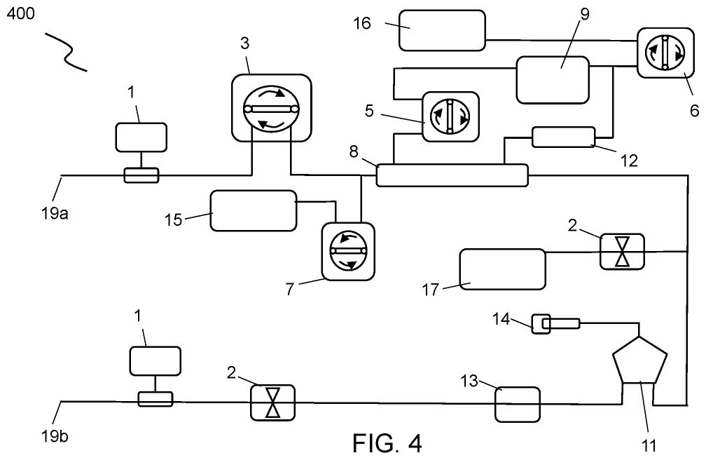

A sorption cartridge according to the present disclosure may also form part of a wearable hemodialysis system wherein the sorption cartridge package is placed in a wearable hemodialysis system. Continuous filtering by the sorption cartridge may permit the volume of dialysate fluid to be reduced to typically 1-2 litres. As described below with reference to FIG. 4, a wearable hemodialysis system may comprise a vascular access tubing system and a unit comprising a small hemofilter system, fluid pump, power, sensors, electronic controls, a facility to place and replace said sorption cartridge package, typically on a daily basis, or more often as needed and a system to dispose of excess water. An additional and optional function of the sorption cartridge may be to release electrolytes and or buffer solutions into the blood.

A sorption cartridge according to the present disclosure may also form part of an existing Continuous Renal Replacement Therapy (CRRT) device such that solutions from the bags containing ultrafiltrate or used dialysis solutions could be circulated through a purification cartridge utilizing this technology so that the solutions could be continuously reused.

A sorption cartridge according to the present disclosure may also form part of an existing portable dialysis machine such that solutions from the bags containing ultrafiltrate or used dialysis solutions could be circulated through such a purification cartridge utilizing this technology so that the solutions could be continuously reused so that much less solution is required to complete a treatment. The use of such a system may further reduce the constraints on fluid use in a portable system so that higher dialysate flow rates may be used to improve clearance rates and reduce required dialysis times or reduce the need for higher blood flow rates.

A sorption cartridge according to the present disclosure may also form part of an existing hemodialysis machines in such a way that the used dialysis solution could be circulated through such a purification cartridge utilizing this technology and back to the dialyser so that the solution could be continuously reused so that much less solution is required to complete a treatment. Used in this way a continuous supply of purified water is not required. Some of the existing fluid management mechanisms of the dialysis machine such as pressure sensors, blood leak detectors and ultrafiltration pumps and metering systems may continue to be used in this mode. This may allow the flexibility to use a regular dialysis machine in situations such as hospital rooms and home therapies where it is difficult or costly to provide a reliable supply of purified water. This capability may also allow dialysis clinics to continue to provide treatment in cases where safe water supply is interrupted such as floods, earthquakes or other natural disasters. The use of existing machines in such situations would enable the staff that work in that clinic to continue to provide the treatment with minimal additional training.

A sorption cartridge according to the present disclosure may, in any embodiment, further comprise means for supplementing the (purified) blood plasma or dialysate fluid with at least one substance selected from the group consisting of minerals such as calcium, sodium, and potassium; anticoagulants; antimicrobial agents and other medicaments.

A sorption cartridge according to the present disclosure may, in any embodiment, further comprise means for selective sorption of middle molecules, vitamins, and minerals such as calcium, sodium, and potassium. The hydrogel may therefore be loaded with a certain amount of minerals, vitamins, or electrolytes and can only absorb a designated amount.

Optionally, a sorption cartridge according to the present disclosure may incorporate other ion exchange systems.

In another aspect, the present invention provides a method for removing toxic substances from the blood using a sorption cartridge according to the present disclosure.

The sorption cartridge of the present disclosure may take the form of a commercially available hollow fiber dialyser where the dialysate compartment is filled with the sorption material described herein.

In an embodiment, other suitable sorbing materials as small particles may be interspersed within the whole or part of hydrogel in order to enhance the sorbing or electrolyte control of the cartridge. Examples of other suitable sorbing materials may be but are not limited to activated carbon particles, nanoclay particles, graphene-based nanostructured particles, zirconium phosphate particles, and hydrous zirconium oxide particles. Additional sorbent elements can be incorporated at any stage in the device, but preferably it is incorporated in the sorption cartridge system.

FIG. 2 illustrates a sorption cartridge, as described herein, in a hemoperfusion system 200. Blood comes into the arterial line, e.g., inlet (19a), and its pressure is sensed by a pressure sensor (1). The blood pump (3) pumps the blood through the bloodline (25). The anticoagulant pump (7) may inject anticoagulant at the required rate from the anticoagulant solution container (15) into the bloodline (25). The fluid removal pump (6) pumps the required amount of ultrafiltrate from the hemofilter (8) portion of the cartridge into the ultrafiltrate bag (16). A hydrogel cartridge (9), e.g., a sorbent cartridge comprising a hydrogel sorbent according to the disclosure herein, is provided. The sorbent containing portion of the hydrogel cartridge (9) adsorbs uremic toxins and modifies electrolytes in the blood. The heating and/or cooling element (20), which may be positioned inside the hydrogel cartridge (9) or external to hydrogel cartridge (9) as part of system 200, maintains the temperature at an optimal level to facilitate efficient toxin adsorption using the reading from the temperature sensor(s) (18) inside the cartridge. An optimum temperature level for blood may be 37° C. to prevent damage to the blood re-entering the body. A heater element and/or cooling element 20 may be positioned inside or proximate to hydrogel cartridge (9). There is a second temperature sensor (18) that measures the temperature of the blood exiting the hydrogel cartridge (9) to ensure that the temperature of the blood is within a safe physiological range. A prime solution bag (17) and clamp (2) are used to fill the circuit with fluid before treatment, return blood at the end of treatment and allow infusion of fluid during the treatment if required. The air removal filter (11) blocks any accumulated air in the blood and allows it to be removed using the attached syringe (14). The ultrasonic air detector (13) detects bubbles of air in the bloodline (25) to be returned to the patient via outlet (19b). When air is detected in the bloodline (25) an alarm will sound and the blood pump (3) will stop so that corrective action may be taken. Blood returned to the patient is monitored by another pressure sensor (1).

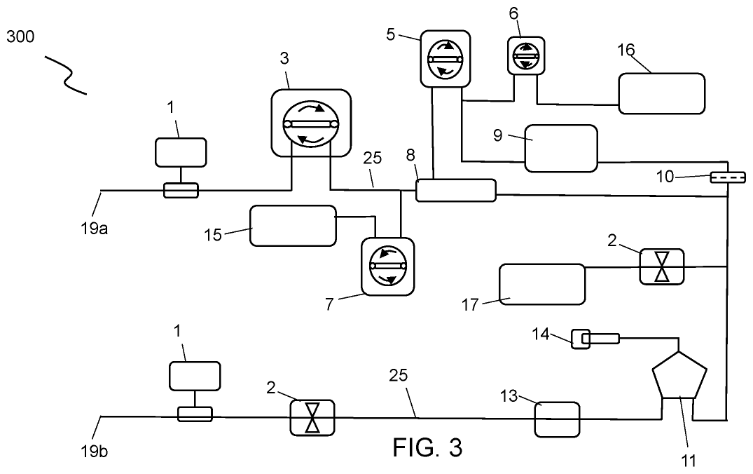

FIG. 3 illustrates a sorption cartridge in a hemofiltration system 300. Blood comes into the arterial line via inlet 19a and its pressure is sensed by a pressure sensor (1). The blood pump (3) pumps the blood through the bloodline (25). The anticoagulant pump (7) injects anticoagulant, e.g. heparin and/or citrate, at the required rate from the anticoagulant solution container (15) into the bloodline (25). The ultrafiltrate pump (4) pumps the required amount of ultrafiltrate from the hemofilter (8) through the hydrogel cartridge (9), then through the reinfusion fluid filter (10) back into the bloodline (25). The reinfusion fluid filter (10) prevents bacteria, endotoxin, and particles from entering the blood. The hydrogel cartridge (9) adsorbs uremic toxins and modifies electrolytes in the blood. The fluid removal pump (6) pumps the required amount of ultrafiltrate from the outlet of the ultrafiltrate pump (4) into the ultrafiltrate bag (16). A prime solution bag (17) and clamp (2) are used to fill the circuit with fluid before treatment, return blood at the end of treatment and allow infusion of fluid during the treatment if required. The air removal filter (11) blocks any accumulated air in the blood and allows it to be removed using the attached syringe (14). The ultrasonic air detector (13) detects bubbles of air in the bloodline (25) to be returned to the patient via outlet 19b. When air is detected in the bloodline (25) an alarm will sound and the blood pump (3) will stop so that corrective action may be taken. Blood returned to the patient is monitored by another pressure sensor (1).

FIG. 4 illustrates a sorption cartridge, as described herein, in a hemodialysis system. Blood comes into the arterial line via inlet 19a and its pressure is sensed by a pressure sensor (1). The blood pump (3) pumps the blood through the bloodline (25). The anticoagulant pump (7) injects anticoagulant at the required rate from the anticoagulant solution container (15) into the bloodline (25). The dialysate pump (5) pumps the required amount of fluid from the hemofilter (8) into and through the hydrogel cartridge (9), then through the blood in dialysate detector (12) back into the hemofilter (8). The hydrogel cartridge (9) adsorbs uremic toxins and modifies electrolytes in the fluid. The fluid removal pump (6) pumps the required amount of ultrafiltrate from the outlet of the hydrogel cartridge (9) into the ultrafiltrate bag (16). A prime solution bag (17) and clamp (2) are used to fill the circuit with fluid before treatment, return blood at the end of treatment and allow infusion of fluid during the treatment if required. The air removal filter (11) blocks any accumulated air in the blood and allows it to be removed using the attached syringe (14). The ultrasonic air detector (13) detects bubbles of air in the bloodline to be returned to the patient. When air is detected in the bloodline (25) an alarm will sound and the blood pump (3) will stop so that corrective action may be taken. Blood returned to the patient via outlet 19b is then monitored by another pressure sensor (1).

FIG. 5 illustrates a sorption cartridge, as described herein, in a peritoneal system. Dialysate fluid comes in from the patient and its pressure is sensed by a pressure sensor (1) to warn of a problem with a blocked peritoneal catheter (not shown) or empty peritoneal cavity. In the outflow cycle, the dialysate pump (5) pumps the dialysate fluid from the pressure sensor (1) through the hydrogel cartridge (9), to the dialysate reservoir (24). The stopped dialysate return pump (26) prevents dialysate circulating from the reservoir (24). The hydrogel cartridge (9) adsorbs uremic toxins and modifies electrolytes in the fluid. The heating and/or cooling element (20) maintains the temperature at an optimal level to facilitate efficient toxin adsorption using the reading from the temperature sensor (18) inside the cartridge. There is a second temperature sensor (18) that measures the temperature of the dialysate fluid exiting the hydrogel cartridge (9) to ensure that the temperature of the dialysate fluid is within a safe physiological range. The infusate pump (21) pumps fluid from the infusate solution container (23) into the dialysate line (27) to restore electrolyte levels and glucose levels as required. In the inflow cycle, the dialysate return pump (26) pumps the dialysate fluid from the dialysate reservoir (24), through the dialysate filter (22) to the pressure sensor (1) to be returned to the patient. The stopped dialysate pump (5) prevents dialysate from circulating back to the hydrogel cartridge (9). Pressure is sensed by the pressure sensor (1) to warn of a blocked catheter. The total fluid pumped by the dialysate return pump (26) in the return cycle can be less than the total amount of fluid pumped by the dialysate pump (5) and the infusion pump (21) in order to create a net fluid removal from the patient. The excess fluid removed from the patient is stored in the reservoir (24).

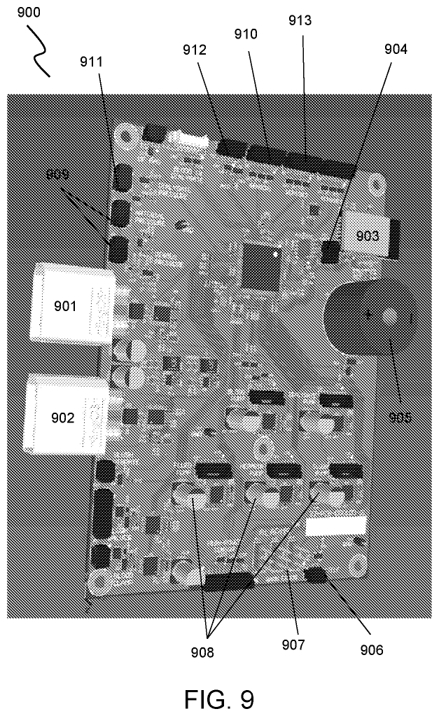

FIG. 9 illustrates an example controller (900) for a renal therapy device according to this disclosure. The renal therapy device may be any embodiment described herein including those described above with respect to FIGS. 2-5. Controller (900) is shown as a circuit board, e.g., a printed circuit board with various electronic components mounted thereto. Controller (900) includes battery input (901) and second battery input (902) for connecting controller (900) to batteries or other power sources that provide power to controller (900) and the renal therapy device when in use. First battery input (901) and second battery input (902) may be electrically isolated so each input (901), (902) may be individually selected so power supply to controller (900) and the renal therapy device is not interrupted, e.g. when one of the batteries in first battery input (901) or second battery input (902) has low power. Controller (900) may be configured to monitor a power charge condition of at least one of the batteries (not shown) coupled to the first battery input (901) and/or the second battery input (902). For example, controller (900) may be configured to monitor a power level, or a voltage level, of a battery coupled to the first battery input (901) and/or the second battery input (902). In some embodiments, having first battery input (901) and second battery input (902) electrically isolated may allow for longer treatment times before power is depleted. When the renal therapy device under control of controller (900) does not need to be used as a portable renal therapy device, controller (900) may receive mains power from one of first battery input (901) and second battery input (902) via an appropriate AC/DC adapter.

Controller (900) may be provided with a data system for storing treatment data of a renal therapy device, according to the present disclosure, for analysis. In an example, controller (900) may be configured to store digital data. In an example, controller (900) may have a digital data storage memory card, SD card, TF card, USB flash drive storage elements, and/or may be configured to communicate with cloud services such as iCloud, Dropbox, Google clouds, or any other digital data servers. In the example illustrated in FIG. 9, the data system comprises an SD card (903). Data system may also comprise a universal asynchronous receiver-transmitter (UART) (904) to allow communication with other devices, e.g. a smartphone or a computer, for transmitting data for analysis and/or storage. UART (904) may include or be coupled to a wireless transceiver for wireless communication with such other devices, e.g., by way of infra-red, Bluetooth, Wi-Fi, or the like.

Controller (900) may also comprise, and/or be coupled to, alarm(s) configured to alert a patient, e.g. visually, audibly, and/or tactilely, of an event whenever patient intervention is required. FIG. 9 illustrates alarm of controller 900 as an audible alarm actuator (905), and illustrates a tactile unit connection (906) configured to couple with an tactile alarm, e.g. a vibration device, to provide an alternative method to communicate the presence of any alarm. Visual alarms (907), e.g. red, yellow, and green status LEDs, may be coupled to controller (900) to provide additional alarm information. Such an alarm may be any of the alarm conditions disclosed herein. Such an alarm may signal a power charge condition of an interconnected battery or other power source. For example, the alarm may alert a patient to connect a new battery to one of the first battery input 901 and the second battery input 902. The alarm may be generated under control of controller (900) to identify which of the first battery input (901) should be connected to a new battery. For example, the alarm may alert a patient that a ultrafiltration bag is full.

In some embodiments, controller (900) may generate a data signal encoding an alarm condition and transmit the data signal to another device (e.g., a smartphone or computer) to present the alarm condition to the patient. The data signal encoding the alarm condition may be transmitted to such other device (or devices) in real-time or near real-time.

In some embodiments, the alarm condition may be encoded for display at a display (1015).

Controller (900) may also comprise connections (908) for communicating with any pump of a renal therapy system according to this disclosure. Controller (900) may comprise a sensor connection for monitoring a pump rotation status, e.g. of blood pump (3). In an embodiment, the sensor connection may be an encoder connection.

Controller (900) may comprise connections for communicating with a clamp, e.g. clamp (2), which may be a solenoid pinch valve located on the outlet (19b). Clamp (2) may be closed to stop return blood flow if power is lost.

Controller (900) may comprise a first connection (909) to a pressure sensor, e.g. pressure sensors (1) on the arterial and venous lines, and/or a second connection (910) for an optional dialysate pressure sensor (not shown) which may be positioned upstream of dialysate pump (5). Controller (900) may also comprise a third connection (911) to blood in dialysate detector (12), a fourth connection (912) for the air detector (13) and a fifth connection (913) for an optional sensor on ultrafiltrate bag (16) to indicate if the ultrafiltration bag is full. Controller may also comprise additional sensor and driver ports to enable additional treatment modalities.

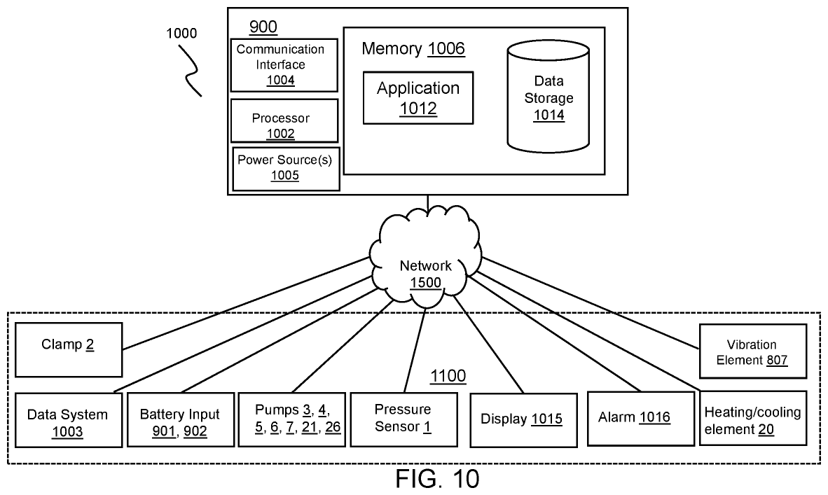

FIG. 10 shows an example system (1000) for controlling a renal therapy device. System 1000 may comprise controller (900), described herein. Controller 900 includes a processor (1002) configured to implement processor readable instructions that, when executed, configure the processor (1002) to conduct operations described herein. The processor (1002) may be a microprocessor or microcontroller, a digital signal processing (DSP) processor, an integrated circuit, a field programmable gate array (FPGA), a reconfigurable processor, a programmable read-only memory (PROM), or combinations thereof. In a non-limiting example micro-controller may be provided by Microchip Technology Part #PIC16F18326T-I/JQ and supplied by Digikey Part #PIC16F18326T-I/JQCT-ND. Controller (900) may be coupled to a plurality of power sources 1005, e.g. battery input (901) and second battery input (902), to provide a portable power supply. Controller (900) may include a communication interface (1004) to communicate with other computing or sensor devices, to access or connect to network resources, or to perform other computing applications by connecting to a network (or multiple networks) capable of carrying data. In some examples, the communication interface (1004) may include one or more busses, interconnects, wires, circuits, and/or any other connection and/or control circuit, or combination thereof. The communication interface (1004) may provide an interface for communicating data between the system (1000) and a display (1015) or an alarm (1016). An alarm described herein, may be any indication provided to a user of the wearable renal therapy device that corrective action should be taken. Non-limiting examples of alarms are visual alerts on a display or a light of the wearable device; vibrations from a vibration actuator of the wearable device; or auditory alerts from a speaker of the wearable device.

Controller (900) may be coupled to at least one of a data system (1003) for storing treatment data of a renal therapy device (e.g. an SD Card port and SD Card, or UART), clamp (2), blood pump (3), ultrafiltrate pump (4), dialysate pump (5), fluid removal pump (6), anticoagulant pump (7), infusate pump (21), dialysate return pump (26), pressure sensor (1), heating/cooling element (20), vibration element (807), display (1015), and/or alarm (1016) via a network (1500). Network (1500) may include any wired or wireless communication path, such as an electrical circuit. In some embodiments, the network (1500) may include one or more busses, interconnects, wires, circuits, and/or any other connection and/or control circuit, or a combination thereof. In some embodiments, the network (1500) may include a wired or a wireless wide area network (WAN), local area network (LAN), a combination thereof, or the like. In some embodiments, the network (1500) may include a Bluetooth® network, a Bluetooth® low energy network, a short-range communication network, or the like.

Controller (900) may include memory (1006). The memory (1006) may include one or a combination of computer memory, such as static random-access memory (SRAM), random-access memory (RAM), read-only memory (ROM), electro-optical memory, magneto-optical memory, erasable programmable read-only memory (EPROM), and electrically-erasable programmable read-only memory (EEPROM), Ferroelectric RAM (FRAM) or the like.

The memory (1006) may store an application (1012) including processor readable instructions for conducting operations described herein. In some examples, the application (1012) may include operations for selecting a power source for a controller and/or renal therapy device according to the present disclosure. A plurality of power sources may be provided, e.g. a first battery input (901) and a second battery input (902), which are electrically isolated to be individually selectable to provide power the controller and/or renal therapy device. Application (1012) may include operations for monitoring a power level of batteries coupled to either battery input (901) or (902). If the power level of a battery coupled to first battery input 901 is above a threshold value, application (1012) may cause the controller and/or renal therapy device to draw power from the battery. If the power level of the battery coupled to first battery input 901 decreases below the threshold value, application (1012) may cause a battery coupled to the second battery input 902 to provide power to the controller and/or renal therapy device. If the power level of all of the plurality of power sources decreases below the threshold value, application (1012) may include operations for closing clamp 2 on an outlet 19b line of the renal therapy device to stop blood flow to a patient.

In another example, application (1012) may include operations to signal an alarm when a power level of a battery coupled a battery input drops below a threshold value. Non-limiting examples of alarms are visual alerts on a display or a light on the renal therapy device; vibrations from a vibration actuator of the renal therapy device; or auditory alerts from a speaker of the renal therapy device. Applicant (1012) may also include operations to signal an alarm when a sensor indicates that air removal filter (11) is full; anticoagulant solution container (15) is empty; ultrafiltrate bag (16) is full; infusate solution container (23) is empty; dialysate reservoir (24) is empty; ultrasonic air detector (13) detects bubbles of air in the bloodline (25); and/or a threshold pressure (e.g. low pressure value) is measured at any one of pumps (3), (4), (5), (6), (7), (21), and (26).

In another example, application (1012) may include operations to signal vibration element (807) to vibrate a sorption cartridge, and hydrogel (806a) and (806b), according the disclosure herein.

In another example, application (1012) may include operations to signal cooling element (805) to create a temperature gradient across a hydrogel of a sorption cartridge according to the disclosure herein.

In another example, application (1012) may include operations to signal a heating element (20) to maintain the process fluid at a desired temperature, e.g. 37° C.

Controller (900) may include a data storage (1014). In some embodiments, the data storage (1014) may be a secure data store. In some embodiments, the data storage (1014) may store received data sets, such as orientation data from air removal filter (11), pressure data, flow rate data, or other types of data. In some examples, the data storage (1014) may store data associated with criteria for analyzing received data sets. In some embodiments, the stored criteria may include pressure criteria that may be used for generating alarms that indications that pressure data may be trending beyond a defined pressure range.

The above description is meant to be exemplary only, and one skilled in the relevant arts will recognize that changes may be made to the embodiments described without departing from the scope of the invention disclosed. The present disclosure may be embodied in other specific forms without departing from the subject matter of the claims. The present disclosure is intended to cover and embrace all suitable changes in technology. Modifications which fall within the scope of the present invention will be apparent to those skilled in the art, in light of a review of this disclosure, and such modifications are intended to fall within the appended claims. Also, the scope of the claims should not be limited by the preferred embodiments set forth in the examples, but should be given the broadest interpretation consistent with the description as a whole.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

As can be understood, the examples described above and illustrated are intended to be exemplary only.

### REFERENCES

D. Niether, S. Di Lecce, F. Bresme and S. Wiegand, Phys. Chem. Chem. Phys., 2017, DOI: 10.1039/C7CP05843H
Mueller B A, Jasiak K D, Thiel S R, et al. Vibration enhances clearance of solutes with varying molecular weights during in vitro hemodialysis. ASAIO Journal (American Society for Artificial Internal Organs: 1992). 2013 March-April; 59(2):140-144. DOI: 10.1097/mat.0b013e3182837ff0.
Kim J, C, Kim J, C, Garzotto F, Cruz D, N, Goh C, Y, Nalesso F, Kim J, H, Kang E, Kim H, C, Ronco C: Enhancement of Solute Removal in a Hollow-Fiber Hemodialyzer by Mechanical Vibration. Blood Purif 2011; 31:227-234. doi: 10.1159/000321073
Hornik, B.; Dutawa, J.; Marcisz, C.; Korchut, W.; Durmata, J. The Effect of Mechanically-Generated Vibrations on the Efficacy of Hemodialysis; Assessment of Patients' Safety: Preliminary Reports. Int. J. Environ. Res. Public Health 2019, 16, 594.
M. J. Story, J. C. R. Turner: Thermal diffusion of diphenyl in benzene and of urea in water. Trans. Faraday Soc., 1969, 65, 1810-1811. DOI: 10.1039/TF9696501810.
B. D. Butler, J. C. R. Turner, Flow-cell studies of thermal diffusion in liquids. Part 1.—Cell construction and calibration. Trans. Faraday Soc., 1966, 62, 3114-3120. doi/10.1039/TF9666203114

The disclosure of the above references are each hereby incorporated herein by reference their entirety.

1. A system for controlling a renal therapy device, the system comprising:
a controller comprising a non-transitory computer readable storage medium having program code stored thereon, the program code executable by a computer, processor or logic circuit to perform a method comprising:
receiving a first power level of a first battery to determine if the first power level of the first battery is above a threshold value;
receiving a second power level of a second battery to determine if the second power level of the second battery is above the threshold value;
when the first power level is above the threshold value, causing the renal therapy device to be powered by the first battery;
when the first power level is below the threshold value, and the second power level is above the threshold value, causing the renal therapy device to be powered by the second battery;
wherein the first battery is electrically isolated from the second battery.

2. The system of claim 1, wherein the renal therapy device is a portable wearable renal therapy device.

3. The system of claim 1, wherein the threshold value is the minimum power required to operate the renal therapy system.

4. The system of claim 1, comprising sending a signal to an alarm when at least one of the first power level and second power level is below the threshold value.

5. The system of claim 1, comprising signaling a clamp to stop process fluid from the renal therapy device when the first power level and second power level are below the threshold value.

6. The system of claim 1, wherein the renal therapy device comprises a sorbent cartridge, the sorbent cartridge comprising a hydrogel configured to absorb or adsorb a toxin from process fluid without use of a dialysate to purify the process fluid.

7. The system of claim 6, comprising sending a signal to a vibration element to vibrate the sorption cartridge and hydrogel.

8. The system of claim 6, comprising sending a signal to a cooling element to create a temperature gradient across the hydrogel of the sorption cartridge.

9. The system of claim 1, comprising signaling a heating element to maintain process fluid at a desired temperature.

10. The system of claim 9, wherein the desired temperature is about 37° C.

11. The system of claim 1, comprising temperature sensors at opposing ends of the hydrogel to measure a temperature gradient across the hydrogel.

12. The system of claim 1, comprising a filtration circuit having an inlet for receiving process fluid and an outlet to return treated process fluid to a patient, the filtration circuit comprising at least one of a process fluid pump; a hemofilter, and an air removal filter; and at least one of a pressure sensor on the filtration circuit, an ultrasonic air detector on the filtration circuit, a blood leak detector coupled to the hemofilter, and a air sensor coupled to the air removal filter.

13. The system of claim 12, comprising signaling an alarm if an alarm condition is detected by the pressure sensor on the filtration circuit, the ultrasonic air detector on the filtration circuit, the blood leak detector coupled to the hemofilter, and the air sensor coupled to the air removal filter.

14. The system of claim 12, comprising closing a clamp on the filtration circuit or stopping the process fluid pump if an alarm condition is detected by the pressure sensor on the filtration circuit, the ultrasonic air detector on the filtration circuit, or the blood leak detector coupled to the hemofilter.

15. The system of claim 1, comprising communicating with a data system for storing treatment data of a renal therapy device.

16. The system of claim 15, comprising a sensor for monitoring a pump rotation status, and storing the pump rotation status in the data system.

17. A method for controlling a renal therapy device, the method comprising:
receiving a first power level of a first battery to determine if the first power level of the first battery is above a threshold value;
receiving a second power level of a second battery to determine if the second power level of the second battery is above the threshold value;
when the first power level is above the threshold value, causing the renal therapy device to be powered by the first battery;
when the first power level is below the threshold value, and the second power level is above the threshold value, causing the renal therapy device to be powered by the second battery;
wherein the first battery is electrically isolated from the second battery.

18. The method of claim 17, wherein the renal therapy device is a portable wearable renal therapy device.

19. The method of claim 17, wherein the threshold value is the minimum power required to operate the renal therapy system.

20. (canceled)

21. The method of claim 17, wherein the first power level of the first battery, and the first power level of the first battery, are received from wireless communication with at least one sensor coupled to the first battery and the second battery.
